(19) United States
(12) Patent Application Publication
(10) Pub. No.: US 2009/0176696 A1
(43) Pub. Date: Jul. 9, 2009

(54) Methods And Compositions For Modulating An Immune Response

(75) Inventors: Kingston Mills, Dublin (IE); Ed Lavelle, Dublin (IE); Sarah Higgins, Dublin (IE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Provost Fellows and Scholars of the College of the Holy and Undivided Trinity of Queen Elizabeth, Dublin (IE)

(21) Appl. No.: 12/297,635

(22) PCT Filed: Apr. 18, 2007
(86) PCT No.: PCT/EP2007/053809

§ 371 Date: Oct. 17, 2008

(30) Foreign Application Priority Data
Apr. 18, 2006 (IE) 2006/0293

(51) Int. Cl. A61K 38/06; A61K 38/07

(52) U.S. Cl. 514/8

(57) ABSTRACT

The present invention provides compositions and methods for the suppression of Th2-mediated immune response. Tracheal cytotoxin is shown to mediate a selective suppression of T helper cell type 2 (Th2)-mediated immune responses. The methods and compositions of the invention are useful for the treatment of Th2-mediated diseases and conditions due to their utility in suppressing Th2-mediated immune responses. The invention further extends to methods for suppressing the production of cytokines, such as IL-4 and IL-5 which contribute to the development of Th2-mediated immune responses.

### FIELD OF THE INVENTION

The present invention provides methods for suppressing T-helper 2 type (Th2)-mediated immune responses. In particular, the present invention relates to the use of muramyl peptide compounds in methods for the inhibition of Th2-mediated immune responses, said methods having utility in the treatment of Th2-mediated diseases and conditions. The compounds and methods of the invention further have utility in methods for suppressing the production of the cytokines interleukin 4 (IL-4) and interleukin 5 (IL-5).

### BACKGROUND TO THE INVENTION

Cells of the innate immune system, especially dendritic cells (DCs), direct the differentiation of naïve CD4⁺ T cells into functionally distinct subsets such as Th1, Th2, IL-17-producing T cells (ThIL-17) or regulatory T (Tr) cells.

Activation of immature dendritic cells through binding of conserved microbial molecules to pathogen recognition receptors (PRRs), such as Toll-like receptors (TLRs) and integrins, is accompanied by dendritic cell maturation and homing to the lymph nodes where the mature dendritic cells present antigen (Ag) to the naïve T cells. Activation of DCs by pathogen derived molecules plays a critical role in regulating the differentiation of naïve CD4⁺ T cells into distinct T cell subtypes. Th1 cells confer protection against intracellular infection but are also associated with inflammatory responses and autoimmune disease.

T helper cell type 2 (Th2) cells protect against extracellular immunogens such as bacteria and parasites through the production of antibodies by B cells. Th2 cells produce cytokines, in particular IL-4, IL-5, IL-6, IL-10 and IL-13 which induce the production of antibodies such as IgE. These secreted cytokines are also involved in the recruitment, proliferation, differentiation and survival of eosinophils. A number of Th2 mediated diseases, such as asthma, allergy and atopic dermatitis involve eosinophilia.

Bordetella pertussis causes a protracted and severe disease, which is often complicated by secondary infection and pneumonia, and can have a lethal outcome in young children. Recovery from infection is associated with the development of B. pertussis-specific Th1 cells and these cells play a critical role in clearance of the bacteria from the respiratory tract. However, antigen-specific Th1 responses in the lung and local lymph nodes are severely suppressed during the acute phase of B. pertussis infection. B. pertussis has evolved a number of strategies to circumvent protective immune responses.

One of the most prominent features of pathology during infection with B. pertussis in both animals and humans is the destruction of the ciliated epithelial cell population from the respiratory mucosa. In 1982, Goldman and co-workers (Goldman et al. 1982) reported that a low molecular weight fraction of B. pertussis culture could duplicate this pathology when added to hamster tracheal organ cultures. The active component in the culture was identified as a 921-Da molecule, called tracheal cytotoxin (TCT). The primary structure of TCT is N-acetylglucosaminyl-1,6-anhydro-N-acetylmuramylalanyl-γ-glutamyldiaminopimelylalanine (Cookson et al. 1989b). The incorporation of muramic acid and diaminopimelic acid residues occurs in peptidoglycan, which provides structural rigidity to the bacterial cell wall. The structure of TCT places it in the muramyl peptide family, a group of structurally related molecules that are responsible for a diverse range of biological activities.

Neisseria gonorrhoeae, which also damages human ciliated cells during gonococcal infection of fallopian tube mucosa, releases a 921-Da molecule that is chemically identical to TCT. More recently, TCT was isolated from the luminous, gram-negative bacterium, Vibrio fischeri. However, the release of TCT is not a general property of gram-negative bacteria, despite the fact they share a common peptidoglycan composition.

B. pertussis TCT causes ciliostasis, ciliated cell destruction within cultured hamster respiratory epithelia and can also inhibit DNA synthesis in isolated cultured hamster tracheal epithelial (HTE) cells. TCT inhibition of HTE proliferation may reflect a secondary effect of TCT on the capacity of respiratory epithelium to regenerate the lost ciliated cell population.

IL-1 has been reported to be involved in TCT toxicity in B. pertussis (Heiss et al 1993a). Recombinant IL-1 causes TCT-like damage to the respiratory epithelium. IL-1 inhibits DNA synthesis by HTE cells and generates B. pertussis-like destruction of epithelial cells in hamster tracheal organ culture. Furthermore, TCT stimulates IL-1 alpha production by respiratory epithelial cells. The IL-1 produced remains intracellular, consistent with the observations that the effects of TCT cannot be blocked using either anti-IL-1 alpha antibodies or the IL-1 receptor antagonist. The toxicity of TCT has been linked to the induction of NOS in response to the production of intracellular IL-1 in the respiratory epithelium (Heiss et al. 1994). TCT and endotoxin have also been found to be highly synergistic in the induction of IL-1alpha (IL-1α), type II iNOS, NO and inhibition of DNA synthesis when added to HTE cells (Flak et al. 2000).

Members of the muramyl peptide family of compounds have been shown to be capable of enhancing T cell and antibody responses to co-administered antigens. It is well established that the adjuvant activity of peptidoglycan is attributed to the muramyl peptide structure, muramyl dipeptide (MDP). A synergistic effect of other muramyl peptides with LPS has also been reported. Yang et al., (Yang et al. 2000) demonstrated that MDP could synergise with LPS or lipoteichoic acid to induce IL-8 production in human monocytic cells. Wang and co-workers (2001) reported that co-administration of MDP with LPS caused significantly increased concentrations of TNF-alpha and IL-6 in cultures of whole human blood, whereas IL-10 production was not influenced. Wolfert et al. (2002) reported that MDP synergises with LPS or peptidoglycan to induce synthesis of TNF-alpha in the human monocytic cell line Mono Mac 6 (Wolfert et al. 2002). Recently, chemically synthesized MDP and several chemically synthesized muramyl peptide derivatives were reported to synergise for TNF-alpha, IL-1 beta, IL-6 and IL-10 production from whole human blood cultures (Traub et al. 2004).

B. pertussis paralyses the ciliary clearance function of the respiratory tract through the release of a 921-Da peptidoglycan fragment, TCT, a component of the bacterial cell wall. The NO-mediated ciliostasis induced by TCT facilitates the survival and replication of B. pertussis in the respiratory tract.

TCT has further been shown to activate an innate defence pathway in the fruit fly, Drosophila melanogaster. Insects depend solely on innate immune responses to survive infection. In Drosophila, the IMD pathway (named after ‘immune deficient’ mutants) is required for antimicrobial gene expression in response to gram-negative bacteria. The IMD pathway is very sensitive to TCT (monomeric) and polymeric gram-negative peptidoglycans. Activation of the IMD pathway was found to require the diaminopimelic acid residue of gram-negative peptidoglycans.

The NOD (nucleotide-binding oligomerization domain) proteins NOD1 and NOD2 have important roles in innate immunity as sensors of microbial components derived from bacterial peptidoglycan. Both NOD1 and NOD2 are mainly expressed by cells of the innate immune system such as antigen presenting cells and epithelial cells. Mutations in the gene that encodes NOD2 (CARD15) occur in a subpopulation of patients with Crohn's disease. Polymorphisms in CARD4 (the gene encoding NOD1) are associated with inflammatory bowel disease and asthma.

The biological activity of TCT has been identified as depending on NOD1, however NOD1 detection of TCT was found to be host-specific, as human NOD1 poorly detected TCT, whereas murine NOD1 did so effectively. Human NOD1 was shown to require the tripeptide (_{L}-Ala-_{D}-Glu-mesoDAP) motif for efficient sensing of peptidoglycan, whereas murine NOD1 was shown to detect the tetrapeptide structure (_{L}-Ala-_{D}-Glu-mesoDAP-_{D}-Ala).

The inventors of the present invention have made the surprising discovery that tracheal cytotoxin (TCT) has immunosuppressive activity and acts to selectively suppress Th2-mediated immune responses. The inventors have therefore identified the utility of the present invention in the treatment of Th2-mediated diseases and conditions, these being conditions where aberrant Th2 responses occur. The inventors have further identified the utility of the present invention in suppressing Th2-mediated immune responses in situations where the Th2 response is compromising the effectiveness of a Th1-mediated response against a disease or condition.

### SUMMARY OF THE INVENTION

According to a first aspect of the present invention there is provided a method for suppressing or inhibiting a T helper cell type 2 (Th2)-mediated immune response, the method comprising;
administering a therapeutically effective amount of a composition comprising tracheal cytotoxin (TCT) of formula I:

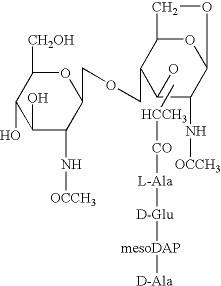

or an analogue or derivative thereof to a subject in need of such treatment.

As herein defined, “tracheal cytotoxin” (TCT) is a specific diaminopimelic acid (DAP) containing muropeptide characterised by the primary structure; N-acetylglucosaminyl-1,6-anhydro-N-acetylmuramylalanyl-γ-glutamyldiaminopimelylalanine (alternatively defined as GlcNAc-(anhydro)MurNAc-L-Ala-D-Glu-mesoDAP-D-Ala).

As herein defined “mesoDAP” relates to meso-diaminopimelate. The term “diaminopimelyl” refers to the incorporation of mesoDAP into the peptide chain.

In certain embodiments, the suppression of the Th2-mediated immune response results in the inhibition or downregulation of at least one cytokine selected from the group comprising: IL-4, IL-5, IL-6, IL-10, and IL-13.

As herein defined, the “suppression” or “inhibition” of a Th2-mediated immune response relates to the lowering of the magnitude of an immune response which is mediated by T helper 2 (Th2) cells. The lowering of the magnitude may result from a reduction of naïve T helper cells differentiating into Th2 type T cells, or from expression of cytokines which drive the differentiation of naïve T helper cells into Th2 cells being reduced, the reduction being a lessening of the amount of Th2 inducing cytokines, such as IL-4, IL-5 and IL-6 over the level which would be present when an Th2 inhibitory compound was not present.

Without wishing to be bound by theory, the inventors predict that in one aspect, the suppression of the Th2-mediated immune response is mediated by TCT suppressing the function of antigen presenting cells (APCs), and particular dendritic cells (DCs), from expression a cytokine profile which results in the differentiation of naïve T helper cells into Th2 type T cells.

As such, in certain embodiments, the methods of this aspect of the invention relate to the administration of a therapeutically effective amount of TCT such that it can couple, bind or otherwise associate with a cell surface activation molecule of at least one type of immune cell, in particular an antigen presenting cell, with this resulting in the prevention, inhibition or down-regulation of one or more functional activities of that cell.

In certain embodiments of the invention, the antigen presenting cell is selected from the group comprising, but not limited to; dendritic cells (DC), follicular dendritic cells, macrophages and B cells.

Furthermore, again without wishing to be bound by theory, the inventors further predict that the immunomodulatory effect mediated by TCT in suppressing the Th2-mediated immune response is further mediated by TCT suppressing the ability of an antigen presenting cell to present antigen.

In certain embodiments of the present invention, wherein the administration of a therapeutically effective amount of TCT suppresses the functional activity of an antigen presenting cell, the antigen presenting cell is a dendritic cell. Said dendritic cell may be an immature dendritic cell, or it may be a mature dendritic cell. Dendritic cells may be myeloid, plasmacytoid, langerhan cells or other dendritic cell subtypes.

In certain embodiments, the subject is a mammal, typically a human.

The present invention further extends to analogues, derivatives, fragments and variants of TCT for use in the present invention. A derivative, fragment or variant of TCT as defined herein is understood to mean any compound, molecule or macromolecule consisting of a portion of TCT which exhibits the observed immunosuppressive properties of TCT. Such derivatives fragments or variants may be prepared by the person skilled in the art using any one of a number of techniques commonly known to the skilled person. Such fragments, variants, analogues or derivatives mediate an identical or substantially similar biological function to that of TCT when acting on the cells of the innate immune system. As such, the present invention is further intended to encompass, in addition to the use of the above listed compounds, the use of homologues and analogues of such compounds. In this context, homologues are molecules having substantial structural similarities to the above-described compounds and analogues are molecules having substantial biological similarities regardless of structural similarities.

Typically a fragment of TCT retains the biological activity of TCT identified herein. For example, the fragment may be a tripeptide comprising the motif: _{L}-Ala-_{D}-Glu-mesoDAP. Alternatively the fragment may be a tetrapeptide comprising the motif: _{L}-Ala-_{D}-Glu-mesoDAP-_{D}-Ala.

In certain embodiments, the TCT is the wild-type TCT molecule derivable from Bordetella pertussis, in particular an active component which is present and obtainable from the culture identified as a 921-Da molecule. The term TCT further encompasses related molecules derived from other bacteria such as Neisseria gonorrhoeae or Vibrio fischeri which have a substantially identical structure and/or biological function. Such molecules may include, but are not limited to, muramyl peptides and other similar proteins obtainable from bacteria with a primary structure substantially homologous to that of TCT.

In certain further embodiments, TCT also encompasses synthetic forms of TCT. For example, a peptidomimetic may be produced based on the peptide sequence of TCT. Furthermore, small molecules or binding fragments, such as antibodies, may be produced which have the same binding specificity to the same epitope as TCT.

In certain further embodiments, the compounds of the invention may be modulated by exchange or substitution of certain amino acid residues. As is well understood, homology at the amino acid level is generally in terms of amino acid similarity or identity. Similarity allows for ‘conservative variation’, such as substitution of one hydrophobic residue such as isoleucine, valine, leucine or methionine for another, or the substitution of one polar residue for another, such as lysine or glutamic acid for aspartic acid, or glutamine for asparagine. Non-peptide mimetics are further provided within the scope of the invention. In certain embodiments, the compounds of the invention can be modified by substituting an alanine (ala, A) residue for a serine (ser, S) residue or a valine (val, V) reside. In certain further embodiments, the glutamic acid (glu, E) residue may be replaced by an aspartate (Asp, D) residue.

As TCT is a low molecular weight compound, which can be purified from B. pertussis and active analogues can be chemically synthesised, it is particularly suitable for commercial development. Furthermore, because of its low molecular weight and structure, it is unlikely that antibody response will be generated against the compound, which offers considerable advantages over existing biological therapeutics for Th2-mediated diseases and conditions.

Modulation of the response of a specific cell type of the immune system can lead, in turn, to a wider modulation of the overall immune response which may be mounted by the cells of the immune system. Accordingly the immunomodulatory activity of TCT described herein causes the suppression of Th2-mediated immune responses.

The invention further extends to compounds which have a structural similarity or identity to TCT for use in suppressing Th2-mediated immune responses. Typically such compounds have conserved biological function in that they are effective in mediating a suppression of Th2-mediated immune responses. The compounds may have primary, secondary or tertiary structural similarity with TCT.

In certain embodiments, the related compound is a tripeptide which comprises the peptide motif L-Ala-D-Glu-mesoDAP. As such, in certain embodiments, the tripeptide may in particular be Tri_{DAP} of formula II:

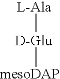

In certain further embodiments, at least one further peptide residue may be added to the tripeptide, this resulting in a peptide comprising at least 4 residues in length. Where the peptide is a tetrapeptide, the compound may be Lactyl-Tetra_{DAP} (OH-HCCH₃-CO-L-Ala-D-Glu-mesoDAP-D-Ala) of formula III:

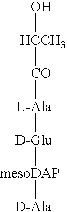

In certain further embodiments, the tetrapeptide may be the TCT analogue FK156 (OH-HCCH₃-CO-L-Ala-D-Glu-mesoDAP-Gly) of formula IV:

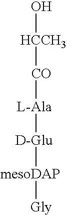

In certain further embodiments, the tetrapeptide may be Tetra_{DAP} (L-Ala-D-Glu-mesoDAP-D-Ala) of formula V:

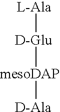

In certain further embodiments, at least one sugar moiety may be attached to the peptide structure to form a murotripeptide or a murotetrapeptide. Typically the sugar is a muramic acid residue. A muropeptide is also known as a muramly peptide. A muramyl peptide is a compound containing one or more sugar residues, at least one of which is typically a muramic acid residue which may be substituted with at least one amino acid residue.

Accordingly in further certain embodiments, the tripeptide may be a murotripeptide such as DAP-containing tripeptide muropeptide. For example, the murotripeptide may be M-Tri_{DAP} of formula VI:

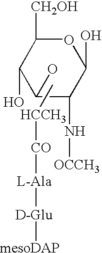

In certain further embodiments, the muropeptide may be a murotetrapeptide, for example, GM-TRI_{DAP} (GlcNAc-MurNAc tripeptide muropeptide).

In certain further embodiments the murotetrapeptide may be M-Tetra_{DAP} of formula VII:

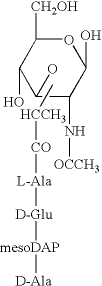

In certain further embodiments, the murotetrapeptide is TCT (Anh-GM-Tetra_{DAP}) as defined hereinbefore as formula I.

In certain further embodiments, the compound may be derived from a compound of formula VIII:

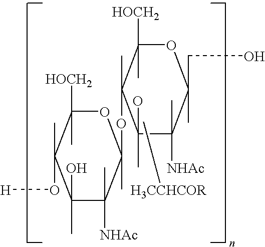

wherein R represents a peptide comprising the motif L-Ala-D-Glu-mesoDAP-D-Ala. In certain further embodiments, R defines a tripeptide comprising the motif: _{L}-Ala-_{D}-Glu-mesoDAP. Alternatively R defines a tetrapeptide comprising the motif: _{L}-Ala-_{D}-Glu-mesoDAP-_{D}-Ala. Typically the peptide is a linear peptide.

Accordingly, in certain further embodiments, the present invention relates to the administration of a composition comprising a diaminopimelic acid (DAP)-containing muropeptide, in an amount sufficient such that said L-Ala-D-Glu-mesoDAP-D-Ala is brought into contact with at least one cell of the innate immune system which is capable of modulating a Th2-mediated immune response or an antigen presenting cell, such that suppression of a T cell mediated immune response results.

A further embodiment of the invention provides for the effective amount of a composition comprising diaminopimelic acid (DAP)-containing muropeptide to couple, bind or otherwise associate with an intracellular or cell surface activation molecule of at least one type of immune cell, this resulting in the prevention, inhibition or down-regulation of one or more functional activities of that cell.

In certain further embodiments the present invention relates to the use of a pharmaceutically acceptable salt of any one of the compounds of the present invention, in particular TCT of formula I. Pharmaceutically acceptable salts are salts that retain the desired biological activity of the parent compound and do not impart undesired toxicological effects. Examples of pharmaceutically acceptable salts are discussed in Berge et al., 1977, “Pharmaceutically Acceptable Salts,” J. Pharm. ScL, Vol. 66, pp. 1-19.

The active compounds disclosed may also be prepared in the form of their solvates. The term “solvate” is used herein in the conventional sense to refer to a complex of solute (e.g., active compound, salt of active compound) and solvent. If the solvent is water, the solvate may be conveniently referred to as a hydrate, for example, a hemihydrate, monohydrate, dihydrate, trihydrate, tetrahydrate, and the like.

The invention further extends to prodrugs of the compounds of the present invention. A prodrug of any of the compounds can be made using well known pharmacological techniques.

The T helper cell type 2 (Th2)-mediated immune response which is suppressed following the administration of the therapeutically effective amounts of the compounds of the present invention (namely, TCT and/or at least one compound having the formula I to VIII) are effective in the prophylaxis and/or treatment of a Th2-mediated disease or condition.

As defined herein, a ‘Th2-mediated immune disease or condition’ means any condition or disease which is mediated in totality or in part by T helper cell type 2 (Th2) T cells. The associated T cell mediated immune response may contribute to the pathogenesis of the disease or condition. In particular a Th2 mediated disease or condition relates to diseases involving immunoglobulin E (IgE) and mast cells due to the development and activation of allergen-specific Th2 cells and it encompasses allergic diseases, such as atopic dermatitis, other dermatological diseases associated with atopy such as; allergic rhinitis or hay fever, allergic bronchial asthma in its acute or chronic, mild or severe forms, with or without acute or chronic bronchitis

Accordingly, the expression “Th2-mediated disease or condition” may further relate to any disease or condition where an aberrant Th2-mediated response occurs. Th2 cell mediated immune responses have been further shown to have implications in the development of conditions such as allergy and asthma.

A “Th2-mediated disease or condition” further includes, but is not limited to: a parasitic infection, a bacterial infection, a fungal infection, inflammatory bowel disease, in particular ulceratice colitis and Crohn's disease, leprosy, systemic lupus erythematosis, Ommen's syndrome, leishmaniasis, toxoplasmosis, trypanosome infection, candiasis and histoplasmosis.

Th2-mediated disease or conditions further extend to type 1 hypersensitivity which comprises common immune disorders, such as, but not limited to; asthma, allergic rhinitis (hay fever), eczema, urticaria (hives) and anaphylaxis. These reactions all involve IgE antibodies which results from the development of Th2 response.

Accordingly a further aspect of the present invention provides a method for the treatment of a Th2-mediated disease or condition, the method comprising:
administering a therapeutically effective amount of a composition comprising at least one compound of formula I to VIII or a derivative or analogue thereof to a subject in need of such treatment.

Accordingly a further aspect of the present invention provides a method for suppressing the production of the cytokine IL-4 and/or the cytokine IL-5, the method comprising:
administering a therapeutically effective amount of a composition comprising at least one compound of formula I to VIII or a derivative or analogue thereof to a subject in need of such treatment.

A yet further aspect of the present invention provides for the use of at least one compound of formula I to VII or a derivative or analogue thereof in the preparation of a medicament for the treatment and/or prophylaxis of a Th2-mediated disease or condition.

In certain embodiments the Th2-mediated condition is asthma, allergy or inflammatory bowel disease.

A yet further aspect of the present invention provides for the use of at least one compound of formula I to VII or a derivative or analogue thereof in the preparation of a medicament for the treatment and/or prophylaxis of a disease which is mediated by increased expression of the cytokine IL-4 and/or the cytokine IL-5.

A yet further aspect of the present invention provides a pharmaceutical composition for the treatment of a Th2-mediated disease or condition, wherein the pharmaceutical composition comprises at least one compound selected from the group comprising formula I to VIII or a derivative or analogue thereof along with at least one pharmaceutically acceptable carrier or diluent.

The inventors have further found that the immunomodulatory effects of TCT or of a composition comprising at least one compound of formula I to VIII in suppressing Th2-mediated immune responses can be enhanced by co-administration of a TLR agonist along with TCT.

Accordingly, a yet further aspect of the present invention provides a method for suppressing a Th2-mediated immune response, the method comprising:
administering a therapeutically effective amount of a composition comprising at least one compound of formula I to VIII or a derivative or analogue thereof, and
administering at least one Toll-like receptor (TLR) agonist, to a subject in need of such treatment.

The Toll-like receptor (TLR) agonist may be administered before, along with or after the administration of the at least one compound of formula I to VIII or a derivative or analogue thereof.

In certain embodiments, the suppression of the Th2-mediated immune response results in the inhibition or downregulation of at least one cytokine selected from the group comprising: IL-4, IL-5, IL-6, IL-10, and IL-13.

In certain embodiments, the TLR agonist is a pharmaceutically acceptable TLR agonist. The TLR agonist may be specific to any defined human Toll-like receptor. In specific embodiments, the TLR agonist has specificity for TLR2, TLR4 or TLR9. In further embodiments the TLR agonist may be selected from any one or more of LPS, CpG motifs, dsRNA, Poly (I:C) and Pam-3Cys.

In a further aspect of the present invention there is provided the use of at least one compound of formula I to VIII or a derivative or analogue thereof along with a TLR agonist in the treatment of a Th2-mediated condition or disease.

In a yet further aspect of the present invention there is provided the use of at least one compound of formula I to VIII or a derivative or analogue thereof along with a TLR agonist in the preparation of a medicament for the treatment of a Th2-mediated condition or disease.

In a still further aspect of the present invention there is provided a pharmaceutical composition for the treatment of a Th2-mediated condition or disease, the composition comprising; at least one compound of formula I to VIII or a derivative or analogue thereof along with a TLR agonist and at least one pharmaceutically acceptable carrier or diluent.

In a further embodiment, the method comprises the further step of administering a TLR agonist along with the composition. The TLR agent may be an agonist to any TLR, however in specific embodiments, the TLR agonist may be specific for TLR2, TLR4 or TLR9. In yet further embodiments the TLR agonist may be selected from any one or more of CpG motifs, dsRNA, Poly (I:C) and Pam3Cys.

A yet further aspect of the present invention provides for the use of a diaminopimelic acid (DAP)-containing muropeptide in the preparation of a medicament for the treatment of a Th2-mediated disease or condition.

In one embodiment the diaminopimelic acid (DAP)-containing muropeptide is a diaminopimelic acid (DAP)-containing tetrapeptide muropeptides such as M-Tetra-_{DAP}, FK156, or Lactyl-Tetra_{DAP}.

In another embodiment the diaminopimelic acid (DAP)-containing muropeptide is a DAP-containing tripeptide or muropeptide such as Tri_{DAP}, M-Tri_{DAP} or GM-TRI_{DAP} (GlcNAc-MurNAc tripeptide muropeptide).

The invention further provides kits for carrying out the therapeutic regimens of the invention. Such kits may comprise, in one or more containers, therapeutically or prophylactically effective amounts of the compositions of the invention in a pharmaceutically acceptable form. Such kits may further include instructions for the use of the compositions of the invention, or for the performance of the methods of the invention, or may provide further information to provide a physician with information appropriate to treating a Th2 mediated condition.

The inventors have further surprisingly observed that the administration of a TCT of formula I or of at least one of the compounds of formulas II to VIII results in the upregulation of Th1 cells, The enhancement of Th1 cells results in an increase in the production of the cytokine interferon gamma (IFN-γ). IFN-γ production suppresses the differentiation of undifferentiated T helper cells into Th2 cells. Accordingly, the methods of the present invention further extend to an indirect mechanism for effecting suppression of Th2 cells, this being mediated by the enhancement of Th1 cell production, which is driven by cytokines such as IFN-γ.

Without wishing to be bound by theory, the inventors further predict that the generation of cytokines such as IL-1 and IL-23 which serve to drive the differentiation of T cells into IL-17 producing T cells, can further serve to suppress the differentiation of undifferentiated T cells into Th2 cells.

Furthermore, without wishing to be bound by theory, the inventors of the present invention believe that the down-regulation of Th2-mediated immune responses which results following the administration of TCT of formula I and related compound, such as those defined by formulas II to VIII is mediated, in part, by the modulation of the activity of antigen presenting cells (APC), and in particular dendritic cells (DC) in inducing a T cell mediated immune response. It is believed that the interaction of TCT with dendritic cells inhibits their function as antigen presenting cells with this in turn prevent antigen display to, and co-stimulation of, T cells. TCT is thought to modulate the activity of antigen presenting cells through the inhibition of MHC class II expression, and/or through the enhancement of TLR-agonist induced IL-10 production. Suppression of Th2-mediated responses may further be mediated by RelB, a member of the NF-kappaB family of transcription factors which is essential for DC maturation and antigen presentation of bone marrow-derived dendritic cells. A recent report showed that RelB is exclusively repressed by NF-kappaB2/p100 in HeLa cells. A report by Speirs et al. (2004) showed that RelB is highly active in NF-kappaB2/p100 knock out (KO) DC. In the absence of NF-kappaB2 DC are hyperactivated, showing increased MHC class II and costimulatory molecule expression, with both being constitutively expressed in response to stimuli. NF-kappaB2 KO DC were also shown to be more efficient (up to 10 times) than wildtype DC in inducing activation of CD4⁺ T cells. It is therefore concluded that NF-kappaB2 was a critical regulator of DC function. TCT may therefore function to prevent the dissociation of NF-kappaB2/p100 from RelB in DC in response to a stimuli such as LPS or Pam-3Csk. Repressed RelB activity would result in decreased ability to induce CD4⁺ T cell response.

TCT may target the MAP kinase pathway in DC. A further alternative is that TCT may sequester MHC Class II molecules intracellularly.

A further potential pathway uses caspases, a large family of serine proteases which use cysteine as the nucleophilic group to cleave substrate at the C terminus of aspartic acid. Caspases have been extensively characterised in the context of their function in apoptosis. However, mammalian caspases have also evolved additional roles in the inflammatory response. More recently, caspases have been implicated in T cell activation. Recently, a study by Wong and co-workers (2004) have demonstrated an additional role for caspases in the regulation of endosomal trafficking pathways that appears to include MHC class II distribution during maturation of DC. In immature bone marrow derived-DC, a number of molecules involved in intracellular trafficking were present in cleaved form, degraded by caspase-like proteases. Cleavage was either inhibited or significantly reduced during maturation of DC induced by either LPS or by peptides that inhibit caspase activity (caspase −1, −3, −4 −7 and −6, −8, −9, −10). Furthermore, treatment of DC with LPS or with certain caspase inhibitors resulted in the expression of MHC class II on the DC surface. The authors concluded that changes in cell surface expression of MHC class II is regulated at least in part by the activities of caspases, inducible NO, and its product NO. A study investigating caspase activity in DC stimulated with TCT may yield an important insight into how TCT interferes with DC activation of T cells.

### DEFINITIONS

As used herein, the term “immune cell” includes cells that are of haematopoietic origin and that play a role in the immune response. Immune cells include lymphocytes, such as B cells and T cells; natural killer cells; myeloid cells, such as monocytes, macrophages, dendritic cells, eosinophils, mast cells, basophils, and granulocytes.

As used herein, the term “T cell” includes CD4+ T cells and CD8+ T cells. The term T cell also includes both T helper 1 type T cells and T helper 2 type T cells and also Th-IL 17 cells.

As used herein, the term “antigen-presenting cell” or “antigen-presenting cells” or its abbreviation “APC” or “APCs” refers to a cell or cells capable of endocytotic adsorption, processing and presenting of an antigen. The term includes professional antigen presenting cells for example; B lymphocytes, monocytes, dendritic cells (DCs) and Langerhans cells, as well as other antigen presenting cells such as keratinocytes, endothelial cells, glial cells, fibroblasts and oligodendrocytes. The term “antigen presenting” means the display of antigen as peptide fragments bound to MHC molecules, on the cell surface. Many different kinds of cells may function as APCs including, for example, macrophages, B cells, follicular dendritic cells and dendritic cells.

As used herein, the term “immune response” includes T cell mediated and/or B cell mediated immune responses that are influenced by modulation of T cell co-stimulation. The term immune response further includes immune responses that are indirectly effected by T cell activation such as antibody production (humoral responses) and the activation of cytokine responsive cells such as macrophages.

As used herein, the term “dendritic cell” or “dendritic cells” (DC) refers to a dendritic cell or cells in its broadest context and includes any DC that is capable of antigen presentation. The term includes all DC that initiate an immune response and/or present an antigen to T lymphocytes and/or provide T-cells with any other activation signal required for stimulation of an immune response. Reference herein to “DC” should be read as including reference to cells exhibiting dendritic cell morphology, phenotype or functional activity and to mutants or variants thereof. The morphological features of dendritic cells may include, but are not limited to, long cytoplasmic processes or large cells with multiple fine dendrites. Phenotypic characteristics may include, but are not limited to, expression of one or more of MHC class I molecules, MHC class II molecules, CD11c, B220, CD8-alpha, CD1, CD4.

As used herein the term “antigen” is any organic or inorganic molecule capable of stimulating an immune response. The term “antigen” as used herein extends to any molecule such as, but not limited, to a peptide, polypeptide, protein, nucleic acid molecule, carbohydrate molecule, organic or inorganic molecule capable of stimulating an immune response.

A “subject” in the context of the present invention includes and encompasses mammals such as humans, primates and livestock animals (e.g. sheep, pigs, cattle, horses, donkeys); laboratory test animals such as mice, rabbits, rats and guinea pigs; and companion animals such as dogs and cats. It is preferred for the purposes of the present invention that the mammal is a human.

It should be understood that the allograft that is transplanted into a host may be in any suitable form. For example, the graft may comprise a population of cells existing as a single cell suspension or it may comprise a tissue sample fragment or an organ. The allograft may be provided by any suitable donor source. For example, the cells may be isolated from an individual or from an existing cell line. The tissue allograft may also be derived from an in-vitro source such as a tissue sample or organ, which has been generated or synthesized in-vitro.

A reduction in the presentation of an allograft antigen to host T cells or host antigen to donor T cells, as processed and presented by DC, has the potential to prevent or limit the extent of an immune response. This reduction in presentation may be achieved by, for example either down-regulation of antigen-processing or reducing or preventing antigen presentation. In this context, a “host” is synonymous with “subject” and includes a human subject, as well as other animals such as other mammals inter alia, as hereinbefore described.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like.

The nomenclature used to describe the polypeptide constituents of the fusion protein of the present invention follows the conventional practice wherein the amino group (N) is presented to the left and the carboxy group to the right of each amino acid residue.

The expression “amino acid” as used herein is intended to include both natural and synthetic amino acids, and both D and L amino acids. A synthetic amino acid also encompasses chemically modified amino acids, including, but not limited to salts, and amino acid derivatives such as amides. Amino acids present within the polypeptides of the present invention can be modified by methylation, amidation, acetylation or substitution with other chemical groups which can change the circulating half life without adversely affecting their biological activity.

The terms “peptide”, “polypeptide” and “protein” are used herein interchangeably to describe a series of at least two amino acids covalently linked by peptide bonds or modified peptide bonds such as isosteres. No limitation is placed on the maximum number of amino acids which may comprise a peptide or protein. The terms “oligomer” and “oligopeptide” are also intended to mean a peptide as described herein. Furthermore, the term polypeptide extends to fragments, analogues and derivatives of a peptide, wherein said fragment, analogue or derivative retains the same biological functional activity as the peptide from which the fragment, derivative or analogue is derived.

### Treatment

As used herein, the term “therapeutically effective amount” means the amount of a compound of the invention which is required to reduce the severity of and/or ameliorate a Th2-mediated disease or condition or at least one symptom thereof, or which serves to prevent the progression of a Th2-mediated disease or condition or one or more of the symptoms associated therewith.

As used herein, the term “prophylactically effective amount” relates to the amount of a composition which is required to prevent the initial onset, progression or recurrence of a Th2-mediated disease or condition or at least one symptom thereof in a subject following the administration of the compounds of the present invention.

As used herein, the term “treatment” and associated terms such as “treat” and “treating” means the reduction of the progression, severity and/or duration of a Th2-mediated disease or condition or the amelioration of at least one of the symptoms thereof, wherein said reduction or amelioration results from the administration of at least one compound of formula I to VIII of the present invention. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

### Administration

TCT or a compound of formula I to VIII or a variant, analogue or fragment thereof for use in the present invention may be administered alone but will preferably be administered as a pharmaceutical composition, which will generally comprise a suitable pharmaceutical excipient, diluent or carrier selected depending on the intended route of administration.

TCT or a compound of formula I to VIII or a variant, analogue or fragment thereof for use in the present invention may be administered to a patient in need of treatment via any suitable route. The precise dose will depend upon a number of factors, including the precise nature of the form of TCT or the compound of formula I to VIII to be administered.

Although the preferred route of administration is parenterally (including subcutaneous, intramuscular, intravenous, by means of, for example a drip patch), some further suitable routes of administration include (but are not limited to) oral, rectal, nasal, topical (including buccal and sublingual), infusion, vaginal, intradermal, intrperintoeally, intrcranially, intrathecal and epidural administration or administration via oral or nasal inhalation, by means of, for example a nebuliser or inhaler, or by an implant.

For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, Lactated Ringer's injection. Preservatives, stabilisers, buffers, antioxidants and/or other additives may be included, as required.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

The composition may also be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919 or European Patent Application No 0,058,481) copolymers of L-glutamic acid and gamma ethyl-L-glutamate (Sidman et al, Biopolymers 22(1): 547-556, 1985), poly (2-hydroxyethyl-methacrylate) or ethylene vinyl acetate (Langer et al, J. Biomed. Mater. Res. 15: 167-277, 1981, and Langer, Chem. Tech. 12:98-105, 1982).

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition (Dec. 15, 2000) ISBN 0-912734-04-3 and Pharmaceutical Dosage Forms and Drug Delivery Systems; Ansel, H. C. et al. 7^{th} Edition ISBN 0-683305-72-7 the entire disclosures of which are herein incorporated by reference.

### Pharmaceutical Compositions

As described above, the present invention extends to a pharmaceutical composition for the suppression of a Th2-mediated immune response wherein the composition comprises at least TCT or a compound of formula I to VIII, or a derivative, fragment, or variant thereof.

Pharmaceutical compositions according to the present invention and for use in accordance with the present invention may comprise, in addition to active ingredient (i.e. TCT or a compound of formula I to VIII), a pharmaceutically acceptable excipient, carrier, buffer stabiliser or other materials well known to those skilled in the art.

Such materials should be non-toxic and should not interfere with the efficacy of the active ingredient. The precise nature of the carrier or other material will depend on the route of administration, which may be, for example, oral, intravenous, intranasal or via oral or nasal inhalation.

The formulation may be a liquid, for example, a physiologic salt solution containing non-phosphate buffer at pH 6.8-7.6, or a lyophilised or freeze dried powder.

### Dose

The composition is preferably administered to an individual in a “therapeutically effective amount” or a “desired amount”, this being sufficient to show benefit to the individual.

As defined herein, the term an “effective amount” means an amount of a composition comprising a compound of formula I to VIII which is necessary to at least partly obtain the desired response, or to delay the onset or inhibit progression or halt altogether the onset or progression of a particular condition being treated.

The amount varies depending upon the health and physical condition of the subject being treated, the taxonomic group of the subject being treated, the degree of protection desired, the formulation of the composition, the assessment of the medical situation and other relevant factors. It is expected that the amount will fall in a relatively broad range, which may be determined through routine trials.

Prescription of treatment, e.g. decisions on dosage etc, is ultimately within the responsibility and at the discretion of general practitioners, physicians or other medical doctors, and typically takes account of the disorder to be treated, the condition of the individual patient, the site of delivery, the method of administration and other factors known to practitioners.

The optimal dose can be determined by physicians based on a number of parameters including, for example, age, sex, weight, severity of the condition being treated, the active ingredient being administered and the route of administration.

A broad range of doses may be applicable. Considering a patient, for example, from about 0.1 mg to about 1 mg of agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

The present invention will now be described with reference to the following examples which are provided for the purpose of illustration and are not intended to be construed as being limiting on the present invention, and further, with reference to the figures.

### BRIEF DESCRIPTION OF THE DRAWINGS

FIG. 1 shows that pre-treatment of bone marrow derived dendritic cells (BMDC) with TCT enhances IL-10 and IL-6 and suppresses IL-12p40 production in response to the TLR-4 agonist, B. pertussis LPS. Bone marrow-derived DC from BALB/c mice were pretreated with TCT (10 μg/ml) for 1, 6 and 12 hours before further stimulation with B. pertussis LPS (Bp LPS; 100, 1000, 10000 pg/ml) for 12 hours. Cytokine and chemokine concentrations were evaluated by ELISA. Values represent means±SD of triplicate samples. *, p<0.05; **, p<0.01; ***, p<0.001 TCT-treated DC versus un-treated DC,

FIG. 2 shows that TCT enhances IL-10 production by dendritic cells in synergy with the TLR4 agonist MPL,

FIG. 3 shows that pre-treatment of BMDC with TCT enhances the production of IL-10 in response to the TLR-9 agonist, CpG. Bone marrow-derived DC from BALB/c mice were pretreated with TCT (10 μg/ml) for 1, 6 and 12 hours before further stimulation with CpG (10, 100, 1000 ng/ml) for 12 hours. Cytokine and chemokine concentrations were evaluated by ELISA. Values represent means±SD of triplicate samples. *, p<0.05; **, p<0.01,

FIG. 4 shows that pre-treatment of BMDC with TCT enhances IL-10 and IL-6 and suppresses IL-12p40 production in response to the TLR-2 agonist, Pam-3Csk. Bone marrow-derived DC from BALB/c mice were pretreated with TCT (10 μg/ml) for 1, 6 and 12 hours before further stimulation with Pam-3Csk (10, 100, 1000 ng/ml) for 12 hours. Cytokine and chemokine concentrations were evaluated by ELISA. Values represent means±SD of triplicate samples. *, p<0.05; **, p<0.01; ***, p<0.001 TCT-treated DC versus un-treated DC,

FIG. 5 shows that TCT downregulates LPS-induced MHC class II expression on dendritic cells. Bone marrow-derived DC from BALB/c mice were cultured for 6 hours with TCT (10 μg/ml) or medium alone, then stimulated with B. pertussis LPS (10 ng/ml, 100 ng/ml) for a further 12 hours and then analysed for surface expression of MHC class II and costimulatory molecule expression by immunofluorescence analysis with Abs specific for MHC Class II (a), CD86 (b) and ICAM-1 (c). i) Medium only and TCT (10 μg/ml), ii) medium only, TCT, TCT and LPS (10 ng/ml) and (iii) medium only, TCT, TCT and LPS (100 ng/ml). Results are presented as mean fluorescence intensity on DC treated with LPS (solid black line), TCT plus LPS (grey shaded area) versus untreated (control) CD11c+ cells (black shaded area),

FIG. 6 shows that pre-injection with TCT 6 hours before immunisation with alum adsorbed KLH reduces antigen-specific T cell proliferation responses in the spleen and local lymph nodes. BALB/c mice were injected subcutaneously (into the flank) with either PBS alone, PBS and KLH (20 μg/mouse) or TCT (10 μg/mouse)/KLH (20 μg/mouse). 14 days later, spleen and lymph node cells were collected. Spleen (A) and lymph node (B) cell suspensions were cultured in the presence of various concentrations of KLH (2, 10, 50 μg/ml) and PMA/anti-CD3 or medium were used as positive and negative controls respectively. Supernatants were collected 72 hours later, fresh medium was added to the wells and cells were incubated overnight. At day 4, 2 μCi of 3H thymidine was added to each well. Plates were incubated for a further 6 hours, and 3H incorporation was determined. Results are the mean responses (±SD) for 5 mice assessed individually in triplicate,

FIG. 7 shows that TCT impairs splenic antigen-specific T cell response induced with KLH adsorbed to alum in vivo. BALB/c mice were injected subcutaneously into the flank with either PBS alone, PBS and KLH (20 μg/mouse) or TCT (10 μg/mouse) and KLH (20 μg/mouse). 14 days later, the spleens were collected. Single spleen cell suspensions cultured in the presence of increasing concentrations of KLH (2, 10, 50 μg/ml) and PMA/CD3 or medium, (positive and negative controls respectively). Supernatants were removed after 3 days and tested for IFN-gamma, IL-10, IL-4 and IL-5 by ELISA. Results are mean (±SD) values for 5 mice per group. *, p<0.05; **, p<0.001,

FIG. 8 shows that TCT impairs antigen-specific T cell response elicited by Alum in the lymph nodes of immunised mice. BALB/c mice were injected subcutaneously as described in FIG. 6. 14 days later, the inguinal lymph nodes were collected. Pooled lymph node cells were cultured in the presence of graded concentrations of KLH (2, 10, 50 μg/ml) and PMA/CD3 or medium, were used as positive and negative controls respectively. Supernatants were removed after 3 days and tested for IFN-gamma, IL-10, IL-4 and IL-5 by ELISA. Results are mean (±SD) values for 5 mice per group,

FIG. 9 shows that TCT does not impair antibody responses to antigen with alum as an adjuvant. BALB/c mice were injected subcutaneously as described in FIG. 6. 14 days after immunization KLH-specific antibody titres was determined by ELISA. Results are expressed as mean antibody titres (±SD) for 5 mice per group,

FIG. 10 shows that TCT impairs antigen-specific IFN-gamma, IL-4 and IL-5 responses in lymph node cells of mice immunised with antigen and LT as an adjuvant. BALB/c mice were injected subcutaneously as described in FIG. 7. 14 days later, the inguinal lymph nodes were collected and lymph node cell suspensions (1×106 cells/ml) cultured in the presence of KLH (2, 10 and 50 μg/ml). Supernatants were removed after 3 days and concentrations of IFN-gamma, IL-10, IL-4 and IL-5 were evaluated by specific ELISA. Results are mean (±SD) values for 5 mice per group. *, p<0.05; **, p<0.01; ***, p<0.001 6 h PBS s.c. v 6 hours TCT s.c,

FIG. 11 shows that TCT does not impair antibody responses to antigen with LT as adjuvant. BALB/c mice were injected subcutaneously as described in FIG. 7. 14 days after immunization KLH-specific antibody titres was determined by ELISA. Results are expressed as mean antibody titres (±SD) for 5 mice per group,

FIG. 12 shows that adoptive transfer of TCT pre-treated DCs impairs KLH-specific T cell proliferation in the lymph nodes. DC were stimulated with KLH (20 μg/ml), or KLH with TCT (10 μg/ml), Pam-3CSK (500 ng/ml) or TCT plus Pam-3CSK. Cells were treated for 2 hours, washed 5 times (to remove all traces of each stimulus) and injected 1×105 cells s.c. into the flank into naïve mice. Cellular responses in local inguinal lymph nodes were assessed 14 days after cell transfer. Lymph node cells from each treatment group were pooled and cultured in the presence of KLH (2, 10 and 50 μg/ml) or PMA/CD3 or medium, as positive and negative controls respectively. Supernatants were collected 72 hours later for cytokine analysis, see FIG. 6, fresh medium was added to the wells and cells were incubated overnight. At day 4, 2 μCi of 3H thymidine was added to each well. Plates were incubated for a further 6 hours, after which cells and 3H incorporation was determined. Results are the mean responses (±SD) for pooled lymph node cells of 5 mice assessed in triplicate, and

FIG. 13 shows that adoptive transfer of TCT pre-treated DCs modulates the antigen-specific cytokine response to Pam-3Csk in vivo. DC were stimulated with KLH (20 μg/ml), or KLH with TCT (10 μg/ml), Pam-3Csk (500 ng/ml) or TCT plus Pam-3Csk. Cells were treated for 2 hours, washed 5 times (to remove all traces of each stimulus) and injected sub-cutaneously into the flank of naïve mice (1×105/mouse). Cellular responses in local inguinal lymph nodes were assessed 14 days after cell transfer. Lymph node cells from each treatment group were pooled and cultured in the presence of KLH (2, 10 and 50 μg/ml) or PMA/CD3 or medium only, as positive and negative controls respectively. Supernatants were removed after 72 hours and concentrations of IFN-gamma, IL-10, IL-4 and IL-5 were evaluated by specific ELISA. Results are for pooled lymph node cells from 5 mice per group assessed in triplicate,

FIG. 14 shows that TCT impairs antigen-specific T cell responses induced with KLH adsorbed to alum in vivo,

FIG. 15 shows that TCT does not inhibit Th1 and Treg responses induced with a TLR agonist,

FIG. 16 shows that TCT inhibits the induction of Th2 cells in vitro,

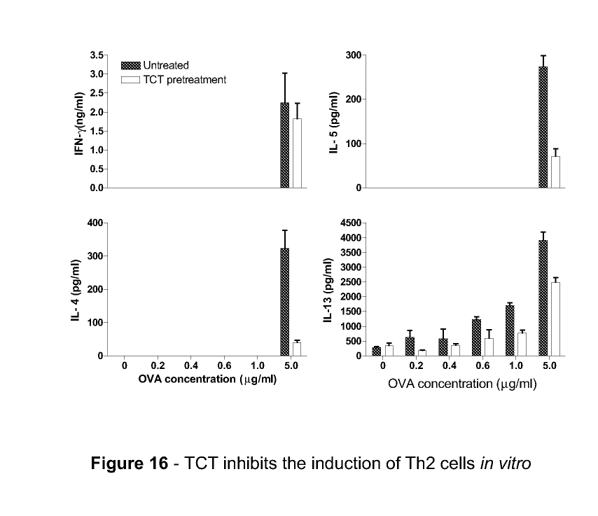

FIG. 17 shows that TCT delays onset of EAE in mice, and

FIG. 18 shows that TCT attenuates Graft versus host Disease in vivo.

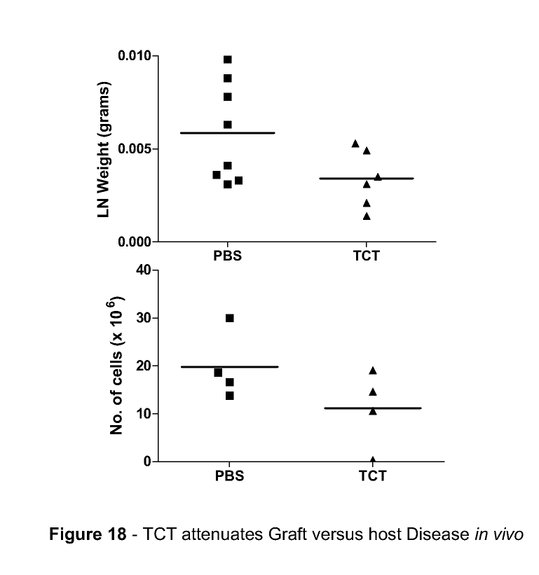

### EXAMPLES

### Example 1

### Pre-Treatment of BMDCs with TCT Significantly Enhances IL-10 and IL-6 Production by TLR2, TLR4 and TLR9 Agonists

### Materials and Methods:

Bone marrow-derived dendritic cells (BMDCs) from BALB/c mice were pre-treated with TCT (10 μg/ml) for 1, 6 and 12 hours before being stimulated with a range of concentrations of the TLR2 agonist, Pam-3Csk (10, 100, 1000 ng/ml), the TLR4 agonist, B. pertussis LPS (100, 1000, 10,000 pg/ml) and the TLR9 agonist, CpG (10, 100, 1000 ng/ml) for a further 12 hours. The concentration range chosen for each TLR agonist was based on preliminary experiments. Cytokine and chemokine concentrations were evaluated by ELISA.

### Results:

Values represent means±SD of triplicate samples. *, p<0.05; **, p<0.01; ***, p<0.001 TCT-treated DC versus un-treated DC.

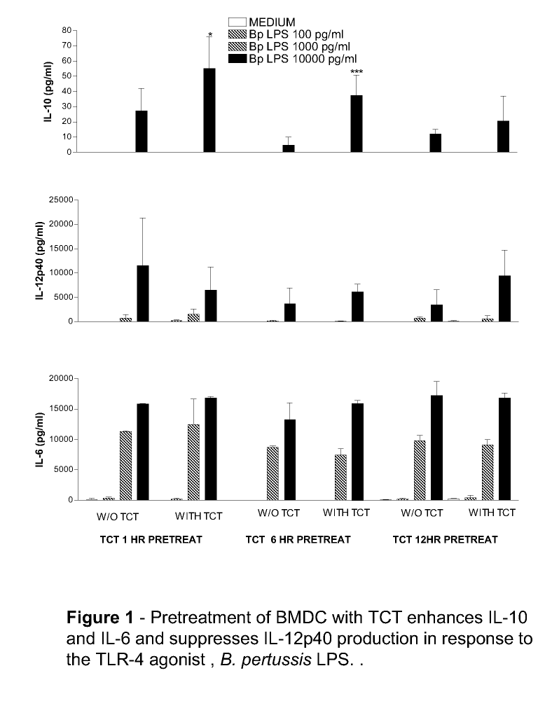

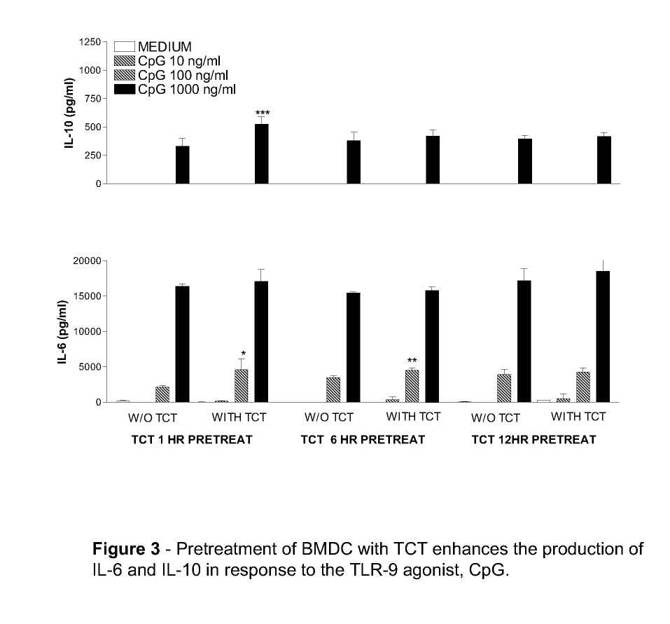

Treatment of DCs with TCT alone did not induce the production of any cytokines and chemokines examined (FIGS. 1, 2, 3 and 4). However, pre-treatment of DCs with TCT (particularly a 1 hour pre-treatment) significantly enhanced the production of IL-10 and IL-6 by all the TLR agonists examined. A significant enhancement of IL-10 production by B. pertussis LPS (10,000 pg/ml) was detected in DCs pre-treated with TCT for 1 and 6 hours (p>0.05, p>0.001) (FIG. 1). Pre-treatment of DCs with TCT significantly reduced IL-12p40 production induced LPS and Pam-3Csk.

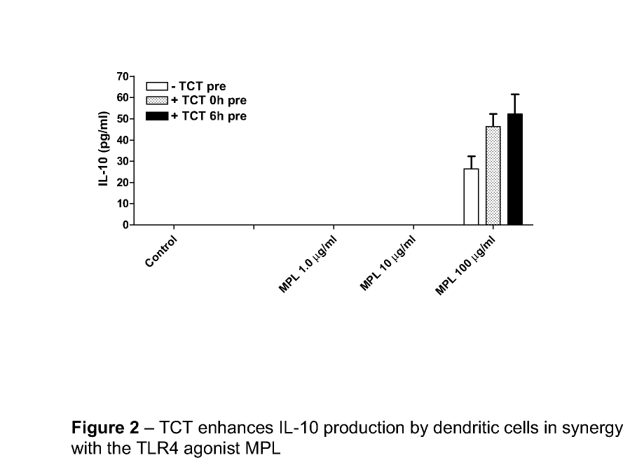

Furthermore, FIG. 2 shows results of experimentation showing that TCT enhances IL-10 production by dendritic cells in synergy with the TLR4 agonist MPL.

Overall, these results indicate that TCT interacts with DCs and enhances anti-inflammatory cytokine production in response to TLR agonists.

### Example 2

### TCT Modulates LPS Induced MHC Class II Expression on DCs

### Materials and Methods:

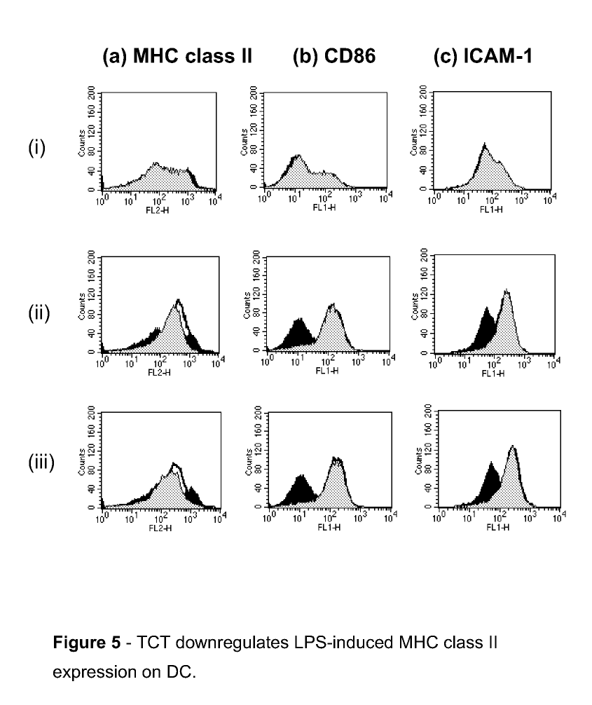

Bone marrow-derived DCs from BALB/c mice were cultured for 6 hours with (i) TCT (10 μg/ml) or medium alone, then stimulated with B. pertussis LPS at (ii) 10 ng/ml and (iii) 100 ng/ml for a further 12 hours and analysed for surface expression of MHC Class II and costimulatory molecules by immunofluorescence analysis with Abs specific for MHC Class II (a), CD86 (b), and ICAM-1 (c). (FIG. 5).

### Results:

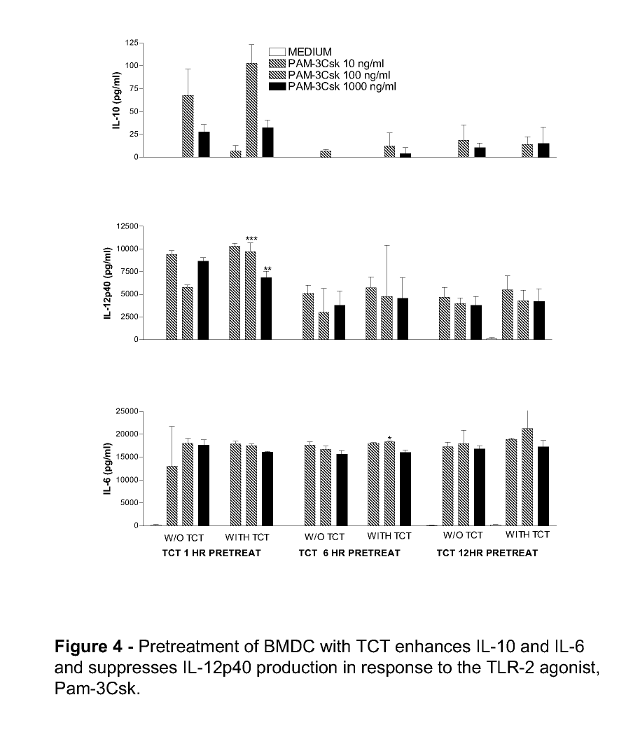

Results are presented in FIG. 4 as mean fluorescence intensity on DCs treated with LPS (solid black line), TCT plus LPS (grey shaded area) versus untreated (control) CD11c⁺ cells (black shaded area).

Stimulation of DCs with TCT (10 μg/ml) alone did not influence MHC Class II or co-stimulatory molecule expression on DCs. In contrast, B. pertussis LPS enhanced expression of the DC surface markers examined.

MHC Class II expression induced with 10 or 100 ng/ml of B. pertussis LPS examined was reduced when DCs were pre-treated for 6 hours with TCT (FIG. 5(a) (ii) and (iii)).

TCT did not modulate B. pertussis LPS induced expression of the other costimulatory molecules examined (FIGS. 5(b) and (c)).

These results indicate that TCT inhibits MHC class II expression may therefore be capable of interfering with the presentation of antigen by DCs to MHC class II-restricted T cells.

### Example 3

### TCT Impairs Antigen-Specific T Cell Responses Elicited by Immunisation with Antigen Adsorbed to Alum

### Materials and Methods:

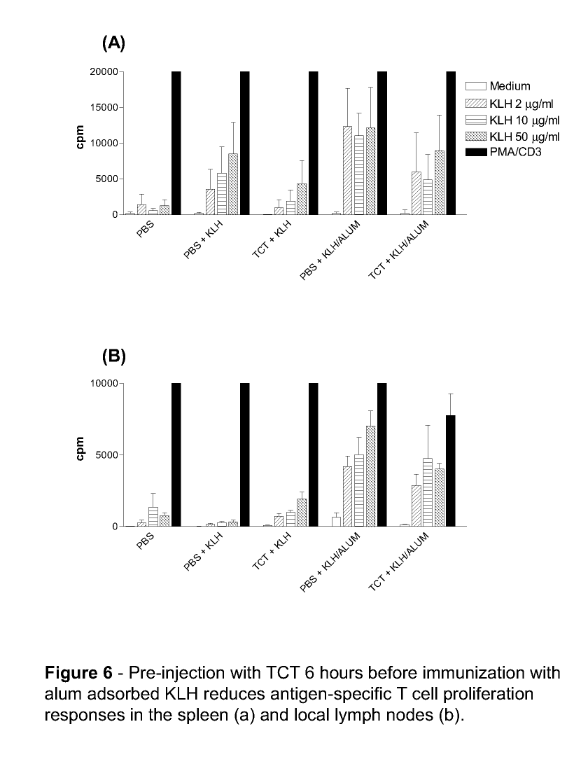

Cohorts of BALB/c mice were injected sub-cutaneously (s.c.) into the flank with PBS or TCT alone, and then immunised sub-cutaneously with KLH or KLH with alum 6 hours later. The adaptive immune response was assessed 14 days later by stimulating spleen and local inguinal lymph node cells with antigen ex vivo. Spleen (FIG. 6A) and lymph node (FIG. 6B) cell suspensions were cultured in the presence of various concentrations of KLH (2, 10, 50 μg/ml) and PMA/anti-CD3 or medium were used as positive and negative controls respectively. Supernatants were collected 72 hours later, fresh medium was added to the wells and cells were incubated overnight. At day 4, 2 μCi of ³H thymidine was added to each well. Plates were incubated for a further 6 hours, and ³H incorporation was determined.

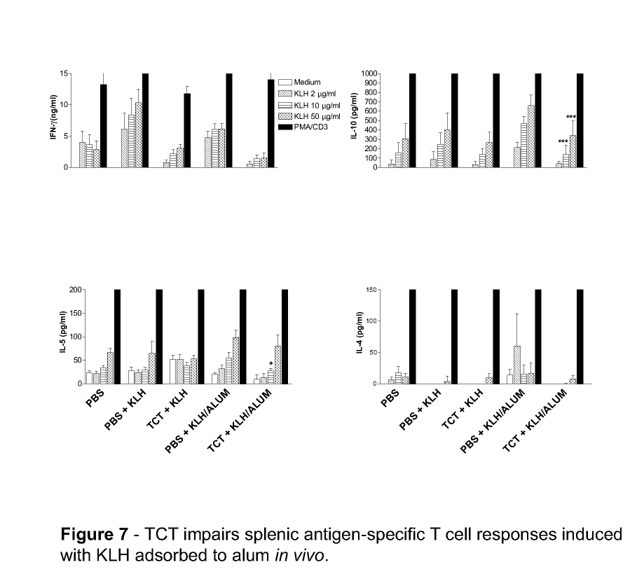

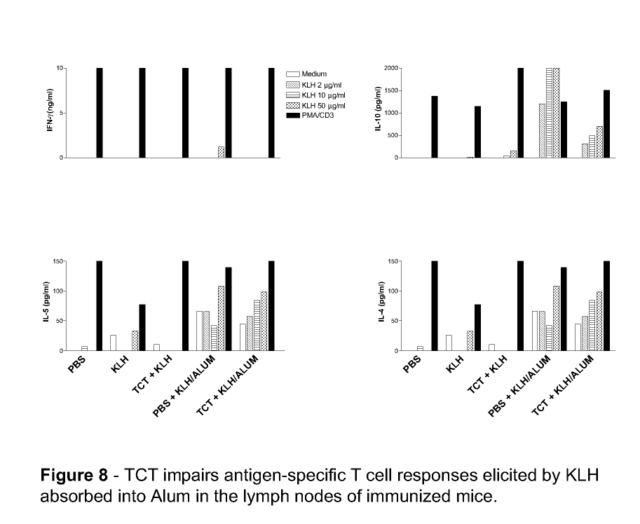

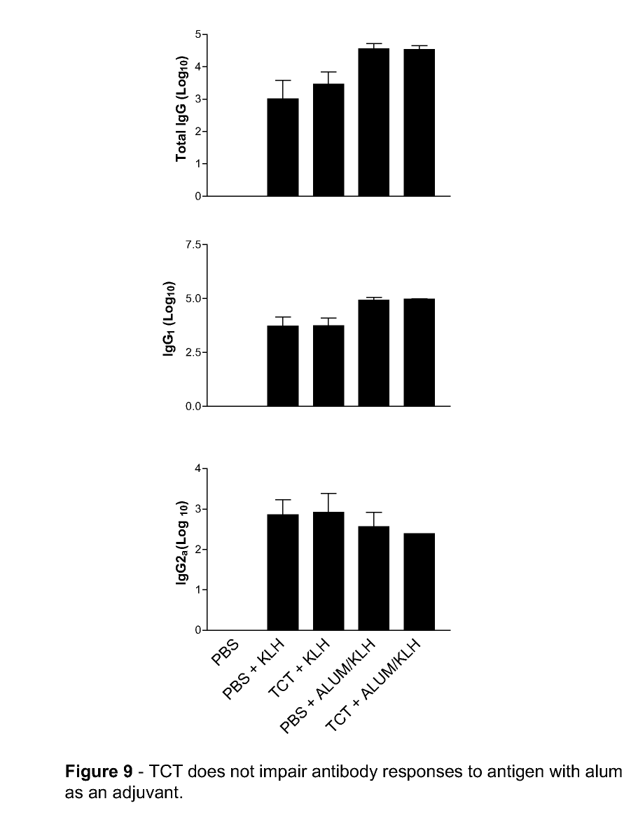

In addition, supernatants were tested for IFN-gamma, IL-10, IL-4 and IL-5 by ELISA (spleen—FIG. 7 and lymph nodes—FIG. 8). KLH-specific antibody titres were also determined by ELISA (FIG. 9).

### Results:

Results are the mean responses (±SD) for 5 mice assessed individually in triplicate.

A reduction in the proliferation response to KLH was observed in spleen and lymph node cells from mice pre-injected with TCT 6 hours before immunisation with alum adsorbed KLH (FIG. 6). TCT also reduced proliferation response in spleen cells from mice immunised with KLH and PBS.

Alum is a widely used clinical adjuvant that promotes the induction of Th2 cells. IL-10, IL-4 and IL-5 production, indicative of a Th2 response, was detected from antigen restimulated spleen cells from mice immunised with KLH and alum (FIG. 7). Treatment with TCT 6 hours prior to immunisation with KLH and alum resulted in a significant reduction in KLH-specific IL-10 production (p<0.001) by spleen cells. IFN-gamma, IL-4 and IL-5 production was also reduced, in spleen cells from mice pre-treated with TCT 6 hours prior to immunisation with followed by KLH and alum (FIG. 7). Notably, the IFN-gamma production was reduced in spleen cells from mice pre-treated with TCT before immunisation with KLH and PBS or immunisation with KLH absorbed to alum (FIG. 7).

Similar to the response detected in the spleen, KLH-specific IL-10 production by lymph node cells from mice pre-treated with TCT 6 hours before immunisation with KLH and alum was severely impaired (FIG. 8). Antigen-specific IL-4 and IL-5 production was reduced in mice pre-treated with TCT 6 hours before immunisation KLH (FIG. 8).

A low concentration of antigen-specific IFN-gamma was detected in lymph node cells from mice immunised with KLH and alum and this was abrogated in mice pre-treated with TCT. Pre-treatment with TCT also attenuated antigen-specific IL-10 in mice immunised with KLH and alum.

Injection of TCT 6 hours before immunisation with KLH only or KLH and alum had no significant effect on total KLH-specific IgG production or on antigen-specific IgG1 and IgG2a titres (FIG. 9).

### Example 4

### TCT Impairs Antigen-Specific T Cell Responses by Lymph Node Cells from Mice Immunised with Antigen and E. coli Heat Labile Enterotoxin (LT) as an Adjuvant

### Materials and Methods:

Groups of 5 BALB/c mice were injected subcutaneously into the flank with PBS or TCT (10 μg/mouse) and then immunised with KLH (20 μg/mouse) or KLH with E. coli heat labile enterotoxin (LT) (100 ng/mouse) 6 hours later. The T cell response was assessed 14 days later from local inguinal lymph node cells stimulated with antigen ex vivo.

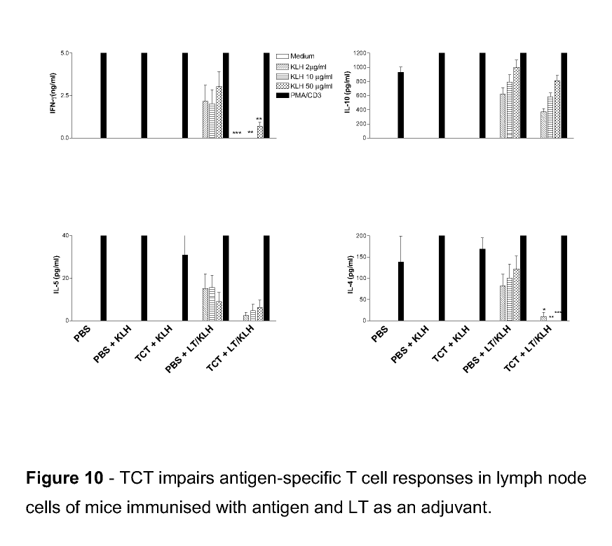

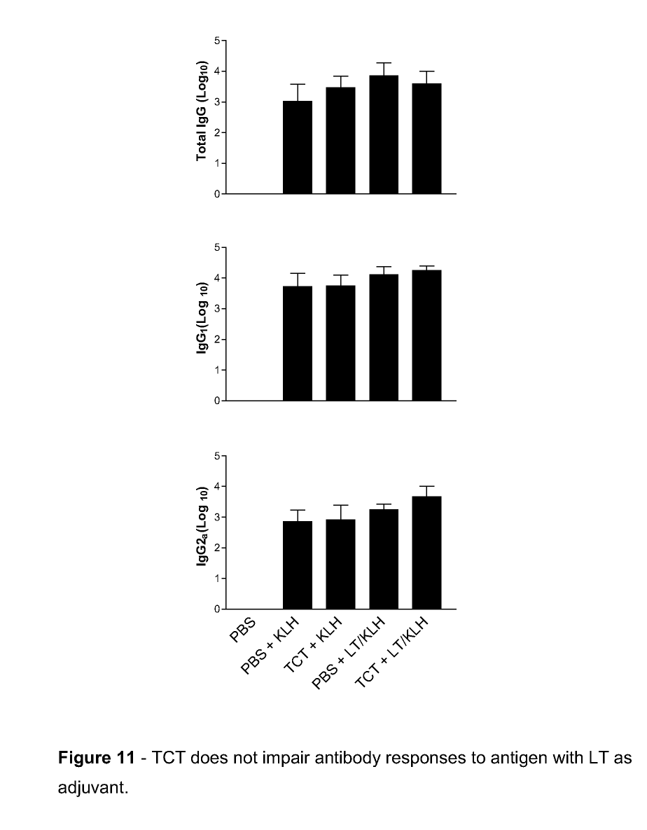

Lymph node (FIG. 10) cell suspensions were cultured in the presence of various concentrations of KLH (2, 10, 50 μg/ml) and PMA/CD3 or medium were used as positive and negative controls respectively. Supernatants were collected 72 hours later. Concentrations of IFN-gamma, IL-10, IL-4 and IL-5 in supernatants were evaluated by ELISA (FIG. 10). KLH-specific antibody titres were also determined by ELISA (FIG. 11). Results are the mean responses (±SD) for 5 mice assessed individually in triplicate.

### Results:

Heat labile enterotoxin (LT) is produced by some enterotoxigenic strains of Escherichia coli, has potent mucosal adjuvant activity and has been used with a wide variety of antigens in animal studies and a number of human clinical trials. LT promotes mixed Th1/Th2 responses. The adjuvant effect of LT has been demonstrated in studies involving immunisation via the subcutaneous, intraperitoneal, intravenous, intradermal and transcutaneous routes.

Results are mean (±SD) values for 5 mice per group. *, p<0.05; **, p<0.01; ***, p<0.001 6 h PBS s.c. v 6 hours TCT sub-cutaneously.

Consistent with previous studies which have shown that LT induced a mixed Th1/Th2 type response, antigen-specific IFN-gamma (IFN-γ), IL-10, IL-4 and IL-5 production was detected in lymph node cells from mice immunised with KLH and LT (FIG. 10).

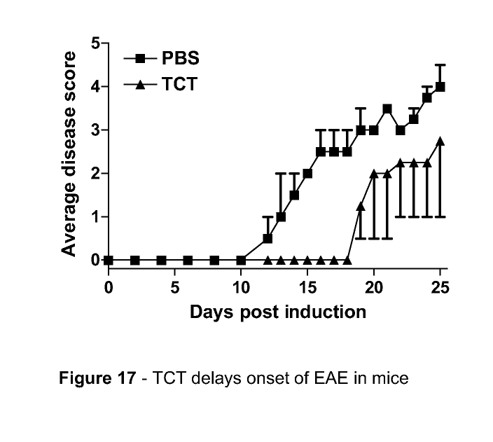

Pre-injection with TCT 6 hours before immunisation with KLH and LT significantly impaired the antigen-specific IFN-gamma and IL-4 production by the lymph node cells (FIG. 17). A reduction in KLH-specific IL-10 and IL-5 production was also evident in lymph node cells from mice injected with TCT 6 hours before immunisation KLH with LT.

Injection with TCT 6 hours before immunisation with LT and KLH had no effect on total KLH-specific IgG production or on antigen-specific IgG1 and IgG2a titres (FIG. 11).

Taken together, these results indicate that injection of TCT 6 hours before immunisation with LT and KLH impairs the antigen-specific T cell cytokine response in the local lymph nodes but does not appear to affect antibody production.

### Example 5

### TCT-Treated DCs Impair Induction of T Cell Responses In Vivo

### Materials and Methods:

Bone marrow-derived DCs from BALB/c mice were cultured in 2% heat inactivated normal mouse sera and 40 ng/ml recombinant GM-CSF for 12 days. DCs were harvested and cultured O/N at a concentration of 1×10⁶ cells/ml. DCs were then incubated with KLH (20 μg/ml) alone, KLH (20 μg/ml) and TCT (10 μg/ml), KLH (20 μg/ml) plus Pam-3Csk (TLR2 agonist; 500 ng/ml), or with KLH (20 μg/ml) and TCT (10 μg/ml) and Pam-3Csk (500 ng/ml). After 2 hours incubation, cells were washed and injected sub-cutaneously (s.c.) into the flank of BALB/c mice (1×10⁵ cells/mouse). T cell responses were assessed 14 days later from pooled local inguinal lymph node cells stimulated with antigen ex-vivo.

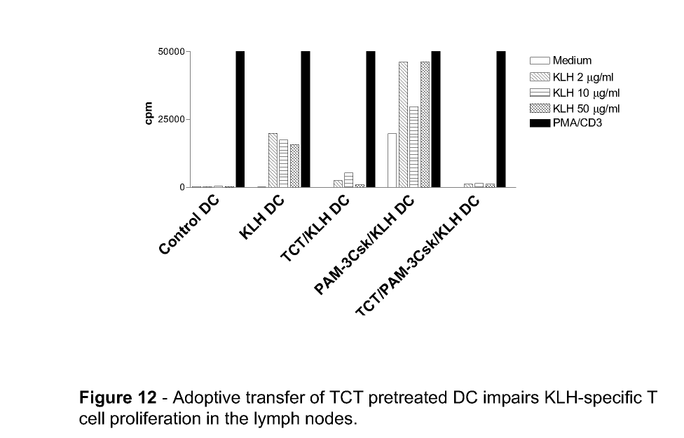

Lymph node cells from each treatment group were pooled and cultured in the presence of KLH (2, 10 and 50 μg/ml) or PMA/CD3 or medium, as positive and negative controls respectively. Supernatants were collected 72 h later for cytokine analysis (IFN-γ, IL-10, IL-4 and IL-5 were evaluated by specific ELISA FIG. 12). Fresh medium was added to the wells and cells were incubated overnight. At day 4, 2 μCi of ³H thymidine was added to each well. Plates were incubated for a further 6 hours, after which cells and ³H incorporation was determined (FIG. 12).

### Results:

Standard protocols to generate murine DCs generally use culture medium supplemented with FCS; however, in vivo transfer of DCs cultured in foetal calf serum (FCS) results in priming of T cells to xenogeneic antigens in the FCS, that complicate the interpretation of DC adoptive transfer experiments. To overcome this problem, normal mouse sera and recombinant GM-CSF were used.

Results are the mean responses (±SD) for pooled lymph node cells of 5 mice assessed in triplicate.

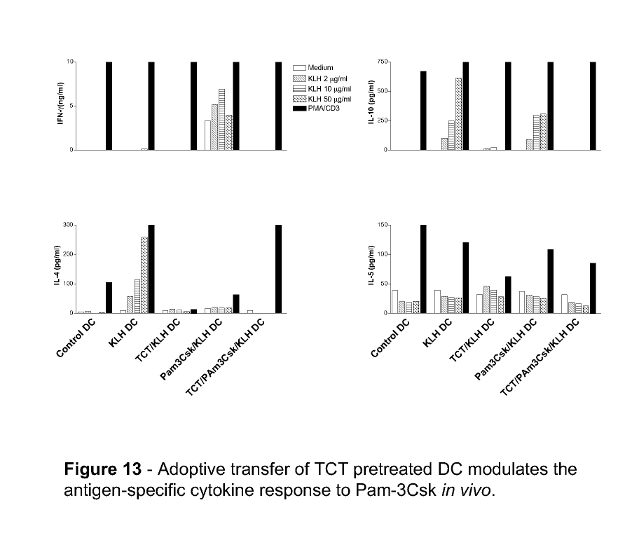

Lymph node cells from mice that received DCs treated with Pam-3Csk with KLH proliferated strongly (FIG. 12). There was a profound impairment of KLH-specific proliferation by lymph node cells from mice that received DCs treated with TCT and Pam-3Csk with KLH (FIG. 13). Furthermore, the moderate proliferation detected by lymph node cells from mice injected with KLH treated DCs alone was almost abolished in mice that received DCs also treated with TCT.

Lymph node cells from mice transferred with DCs treated with KLH and Pam-3Csk produced moderate concentrations of IFN-gamma (IFN-γ) and IL-10 (FIG. 13). This antigen-specific IFN-gamma and IL-10 production was completely abolished in mice that received DCs also treated with TCT (FIG. 13). High concentrations of antigen-specific IL-10 and IL-4 were detected from lymph node cells from mice injected with KLH treated DCs, production of these cytokines was abolished in mice transferred with DCs that had also been treated with TCT. Antigen-specific IL-5 production was not above background concentrations in LN cells from all treatment groups. No KLH-specific IFN-gamma, IL-10 and IL-4 production was detected in lymph node cells from mice that received DCs treated with TCT and KLH.

### Example 6

### Influence of TCT on T Cell Responses In Vivo

### Materials and Methods

BALB/c mice were injected subcutaneously into the footpad with either PBS alone, KLH (20 μg), KLH adsorbed to alum, KLH+LPS (10 μg) or KLH+TCT (10 or 25 μg), KLH+LPS+TCT or KLH+TCT adsorbed to alum. 5 days later mice were sacrificed and the popiliteal LNs were removed. Single LN cell suspensions were cultured in the presence of increasing concentrations of KLH (2-50 μg/ml) or PMA/CD3 or medium as positive and negative controls respectively. Supernatants were taken 3 days later and tested for IFN-γ, IL-10, IL-4 and IL-5 by ELISA. Results are mean (+/−SD) values for 5 mice per group.

### Results:

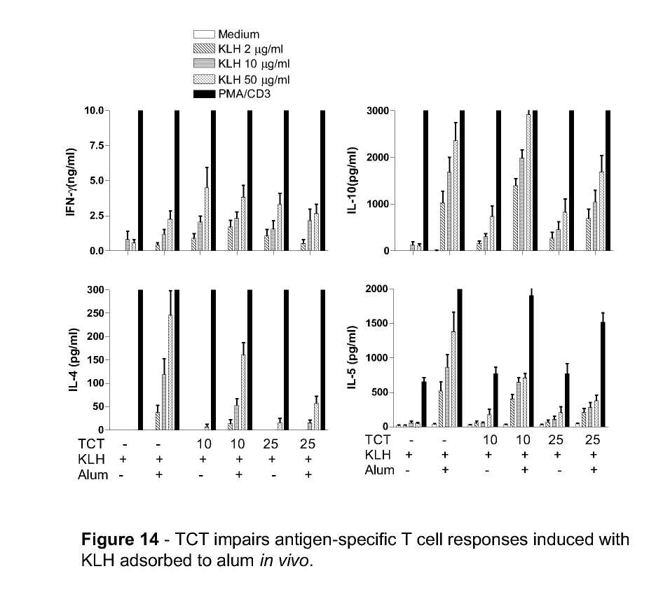

Alum is a widely used clinical adjuvant that promotes the induction of Th2 cells. Consistent with this we found that immunization of mice with KLH in alum generate T cells in the draining lymph nodes that secreted IL-10, IL-4 and IL-5 and low levels of IFN-γ in response to antigen-stimulation in vitro (FIG. 14). The Th2 arm (IL-4 and IL-5) of this response was significantly reduced in mice pre-treated with TCT (10 or 25 μg/mouse). In contrast, IFN-γ and IL-10 production was not significantly affected by treatment with TCT. Furthermore, co-administration of TCT enhanced IFN-γ and IL-10 responses in mice immunized with KLH in PBS. This suggests that TCT may have a specific affect on Th2 cells, while sparing or enhancing Th1 and Treg responses.

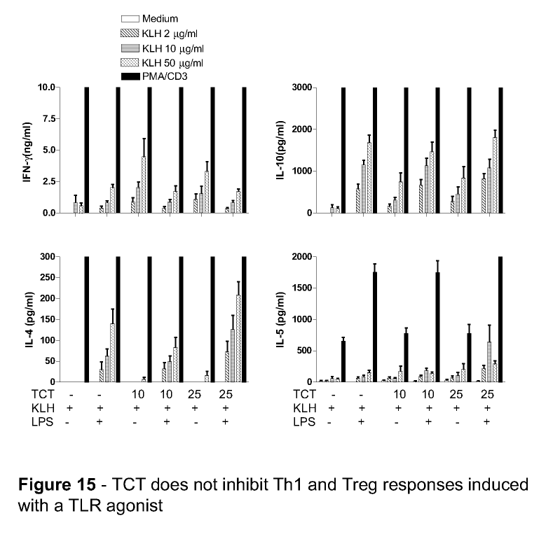

Toll-like receptor (TLR) agonists induce Th1 responses and we have recently demonstrated that they also induce IL-10-secreting Treg cells. Therefore, we also examined the influence of TCT on antigen-specific responses promoted with the TLR against, LPS. Mice were immunized with KLH or KLH and LPS in the presence or absence of TCT. The results (FIG. 15) show that LPS enhanced IFN-γ and IL-10 production, but had little affect on IL-4 or IL-5. These responses were not affected by co-administration of TCT. Furthermore co-administration of TCT with KLH in PBS (without LPS) enhanced IL-10 and IFN-γ production. This confirms that TCT may have a selective inhibitory effect on Th2 type responses.

### Example 7

### TCT Modulates the Induction of T Cell Responses In Vitro

### Materials and Methods:

Dendritic cells act as antigen presenting cells and also serve to direct the induction of distinct T cell responses. Therefore, we examined the influence of TCT on dendritic cells and their ability to promote T cell responses in vitro. Bone marrow-derived dendritic cells DC (BMDC) were cultured with CD4⁺ T cells (4×10⁵) from OVA T cell receptor (TCR) transgenic (Tg) mice. BMDC were pre-treated with TCT (10 μg/ml) or medium only for 2 hours prior to the addition of antigen (OVA 0.2-5 μg/ml) and CD4⁺ T cells purified from the spleens of OVA TCR Tg mice. OVA-specific cytokine production was measured in supernatant taken after 4 days by ELISA.

### Results:

T cells from OVA TCR Tg mice secreted IFN-γ, IL-5, IL-4 and IL-13 in response to OVA-pulsed BMDC. Pre-treatment of DC with TCT significantly reduced OVA-specific IL-4, IL-5 and IL-13 production, but did not affect OVA-specific IFN-γ production. These results indicate that TCT modulates the ability of DCs to promote Th2 type responses in-vitro and are consistent with the effect of TCT in-vivo.

### Example 8

### Influence of TCT on Experimental Autoimmune Encephalomyelitis (EAE)

Experimental autoimmune encephalomyelitis (EAE) is an autoimmune disease of the central nervous system and a murine model for multiple sclerosis. Animals immunised with myelin oligodendrocyte glycoprotein (MOG) with complete Freund's adjuvant (CFA) develop EAE. CD4⁺ T cells, especially IL-17-secreting T cells, mediate the inflammatory pathology in the CNS during the development of EAE.

### Materials and Methods:

EAE was induced in C57BL/6 mice by subcutaneous immunisation with 150 μg MOG peptide 35-55 emulsified in CFA supplemented with 5 mg/ml of killed Mycobacterium tuberculosis. Mice were injected intraperitoneally (i.p) with 500 ng of pertussis toxin (PT) on days 0 and 2. Mice were injected with either PBS or TCT (10 μg/mouse) s.c in the flank on day 1 post EAE induction and every 3-4 days thereafter (FIG. 15). Mice were observed daily for signs of clinical disease. Disease severity was recorded as follows: grade 0, normal; grade 1, limp tail; grade 2, wobbly gait; grade 3, hind limb weakness; grade 4, hind limb paralysis and grade 5, tetraparalysis/death.

### Results:

Untreated mice developed clinical signs of EAE after 12 days and reached grade 3 after 18 days (FIG. 17). In contrast, TCT treated mice did not show any clinical signs of disease until 18 days post induction and the severity of disease was lower than that observed in the untreated control mice (FIG. 17).

### Example 9

### Influence of TCT on Graft Versus Host Disease In Vivo

### Materials and Methods:

Graft-versus-host disease (GVHD) is a major life-threatening complication of bone marrow transplantation, where T cells from the donor graft attack host cells leading to a condition that is only treatable using potent immunosuppressive drugs. It is possible to examine the potential of therapies for the prevention or treatment of GVHD in humans by testing their efficacy using a simple GVHD model in mice. The parent-to-F1 hybrid GVHD murine model involves the transfer of parental lymphocytes into non-conditioned F1 hybrid mice. Using this strain combination the host T cells cannot actively resist the donor cells.

GVHD was induced by transfer of 0.5×10⁷ spleen cells from donor C57BL/6 mice into the footpads of BALB/c×C57Bl/6 F1 hybrid mice. Recipient mice were injected s.c into the footpad with PBS or TCT (10 μg/mouse) 2 hours prior to induction of GVHD. After 7 days, the popliteal lymph nodes were removed, weighed and cell numbers were recorded.

### Results:

The results showed that TCT significantly reduced the weight and the total cells counts in the lymph node of the recipient mice.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention. Reference to any prior art in this specification is not, and should not be taken as, an acknowledgment or any form of suggestion that this prior art forms part of the common general knowledge in any country.

### REFERENCES

Goldman, W. E. et al. 1982. Infect Immun 36:782.
Cookson, B. T. et al. 1989b. Biochemistry 28:1744.
Heiss, L. N. et al. 1993a. Infect Immun 61:3123.
Heiss, L. N et al. 1994. Proc Natl Acad Sci USA 91:267.
Flak, T. A. et al. 2000. Infect Immun 68:1235.
Yang, S. et al. 2001. Infect Immun 69:2045.
Wang, J. E. et al. 2001. Shock 16:178.
Wolfert, M. A. et al. 2002. J Biol Chem 277:39179.
Traub, S. et al. 2004. J Biol Chem 279:8694.
Lemaitre, B. M. et al. 1995. Embo J 14:536.
Kaneko, T. et al. 2004. Immunity 20:637.
Girardin & Philpott, 2004 Mol Immunol. 41:1099
Magalhaes, J G et al. EMBO Reports (2005) 6.1201-1207
Spiers, K et al. 2004 J. Immunol 172:752
Wong S et al. 2004 PNAS 101:17783

1. A method for the treatment and/or prophylaxis of a Th2-mediated disease or condition, the method comprising
administering a therapeutically effective amount of a composition comprising tracheal cytotoxin (TCT) of formula I: 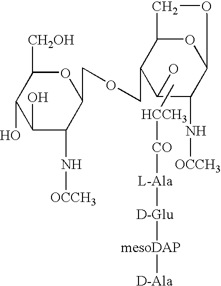
or an analogue or derivative thereof to a subject in need of such treatment.

2. A method as claimed in claim 1 wherein the composition results in the suppression of at least one cytokine selected from the group consisting of IL-4, IL-5, IL-6, IL-10, and IL-13.

3. A method as claimed in claim 1 wherein the subject is a mammal.

4. A method as claimed in claim 3 wherein the mammal is a human.

5. A method as claimed in claim 1 wherein the Th2-mediated disease or condition is selected from the group consisting of asthma, allergy, inflammatory bowel disease, atopic dermatitis, infectious mononucleosis and systemic lupus erythematosis.

6. A method as claimed in claim 1 wherein the Th2-mediated disease or condition is a bacterial condition.

7. A method as claimed in claim 1 wherein the Th2-mediated disease or condition is a parasitic condition.

8. A method as claimed in claim 1 wherein the Th2-mediated disease or condition is a fungal condition.

9. The method as claimed in claim 1 further comprising the step of administering at least one Toll-like receptor (TLR) agonist.

10. The method of claim 9 wherein the Toll-like receptor agonist is selected from the group consisting of a CpG motif, dsRNA, Poly (I:C) and Pam3Cys.

11. A method for suppressing a T helper cell type 2 (Th2)-mediated immune response, the method comprising
administering a therapeutically effective amount of a composition comprising at least one peptide which comprises the peptide motif L-Ala-D-Glu-mesoDAP to a subject in need of such treatment.

12. A method as claimed in claim 11 wherein the peptide is the tripeptide-Tri_{DAP} of formula II:

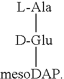

13. A method as claimed in claim 11 wherein the peptide is the tetrapeptide Lactyl-Tetra_{DAP} (OH-HCCH₃-CO-L-Ala-D-Glu-mesoDAP-D-Ala) of formula III:

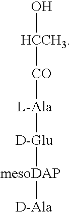

14. A method as claimed in claim 11 wherein the peptide is the tetrapeptide FK156 (OH-HCCH₃-CO-L-Ala-D-Glu-mesoDAP-Gly) of formula IV:

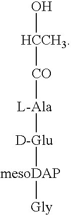

15. The method as claimed in claim 11 wherein the peptide is the tetrapeptide is Tetra_{DAP} (L-Ala-D-Glu-mesoDAP-D-Ala) of formula V:

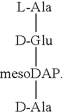

16. The method as claimed in claim 11 wherein at least one sugar moiety is conjoined to the peptide structure to form a muropeptide (muramyl peptide).

17. The method as claimed in claim 16 wherein the muropeptide is M-Tri_{DAP} of formula VI:

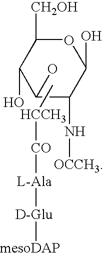

18. The method as claimed in claim 16 wherein the muropeptide is GM-TRI_{DAP} (GlcNAc-MurNAc tripeptide muropeptide).

19. The method as claimed in claim 16 wherein the muropeptide is M-Tetra_{DAP} of formula VII:

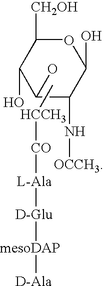

20. The method as claimed in claim 16 wherein the muropeptide is TCT (Anh-GM-Tetra_{DAP}) of formula I:

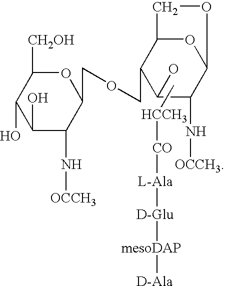

21. The method as claimed in claim 16 wherein the muropeptide a compound of formula VIII: 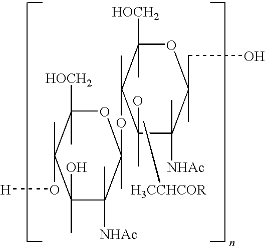
wherein R represents a peptide comprising the motif L-Ala-D-Glu-mesoDAP-D-Ala.

22. The method as claimed in claim 11 further comprising the step of administering at least one Toll-like receptor (TLR) agonist.

23. The method of claim 22 wherein the Toll-like receptor agonist is selected from the group consisting of a CpG motif, dsRNA, Poly (I:C) and Pam3Cys.

24. A method for the treatment of a Th2-mediated disease or condition, the method comprising
administering a therapeutically effective amount of a composition comprising at least one compound of formulas I to VIII or a derivative or analogue thereof to a subject in need of such treatment.

25-26. (canceled)

27. A pharmaceutical composition for the treatment of a Th2-mediated disease or condition, wherein the pharmaceutical composition comprises at least one compound selected from the group consisting of formulas I to VIII or a derivative or analogue thereof along with at least one pharmaceutically acceptable carrier or diluent.

28. A pharmaceutical composition as claimed in claim 27 further comprising at least one Toll-like receptor (TLR) agonist.

29. A pharmaceutical composition as claimed in claim 28 wherein the Toll-like receptor agonist is selected from the group consisting of a CpG motif, dsRNA, Poly (I:C) and Pam3Cys.
